(19) United States
(12) Patent Application Publication
(10) Pub. No.: US 2025/0304541 A1
(43) Pub. Date: Oct. 2, 2025

(54) METHYLENE BLUE HAVING A REDUCED CONTENT OF N-NITROSO-AZURE B IMPURITY AND PROCESS FOR OBTAINING THE SAME

(71) Applicant: ICROM S.r.l., Concorezzo (MB) (IT)

(72) Inventors: Daniele De Zani, Concorezzo (MB) (IT); Simone Rota, Concorezzo (MB) (IT); Anastasiia Tkachuk, Concorezzo (MB) (IT)

(73) Assignee: ICROM S.r.l., Concorezzo (MB) (IT)

(21) Appl. No.: 18/616,250

(22) Filed: Mar. 26, 2024

(51) Int. Cl. C07D 279/20; A61K 31/5415; G01N 30/72

(52) U.S. Cl. CPC C07D 279/20; A61K 31/5415; G01N 30/7233

(57) ABSTRACT

A highly pure methylene blue is disclosed, as well as a process for preparation and a pharmaceutical composition thereof. In particular, the invention relates to highly pure methylene blue essentially free of impurity N-Nitroso-Azure B. It is also described a methylene blue essentially free of other impurities, such as N-nitrosamines and potentially genotoxic impurities. Additionally it is disclosed the use of said highly pure methylene blue in the treatment of methemoglobinemia. Moreover, it is disclosed a method of analysis to determine the level of N-nitroso-Azure B in the methylene blue or in a pharmaceutical composition comprising the same.

### FIELD OF THE INVENTION

The present invention relates to highly pure methylene blue, process for preparation and pharmaceutical composition thereof. In particular, the invention relates to highly pure methylene blue essentially free of N-Nitroso-Azure B. The invention also relates to the use of said highly pure methylene blue in the treatment of methemoglobinemia. Moreover, it is disclosed a method of analysis to determine the level of N-nitroso-Azure B in the methylene blue or in a pharmaceutical composition comprising the same.

### BACKGROUND ART

Colorants having a diaminophenothiazinium structure are well known. Among them, the best known is methylthioninium chloride (3,7-bis(dimethylamino)-5-phenothiazinium chloride) commonly known as methylene blue and used in an increasing number of medical applications in addition to its historical usage. One of its possible resonance structures is depicted below:

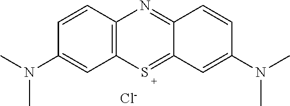

In the medical field, methylene blue is used as an antidote in poisoning by cyanide, methemoglobinemia-inducing agents (such as phenacetin, nitrites, aniline, sulfonamides), and carbon monoxide. It is also used as a chromodiagnostic or chromoendoscopy agent. Recently, its use has been proposed for the treatment of neurodegenerative diseases, viral infections, bipolar disorders and as a tracer of the lymphatic system.

The original process for the synthesis of methylene blue was developed in 1877 (German patent number 1886, Badische Anilin-und Soda Fabrik). Over the years, the process has been revised and improved, but it is still based on the use of a large number of reagents and metal catalysts based on iron, chromium, zinc, aluminium, copper, manganese and zinc. The methylene blue contamination by metal species is therefore one of the issues and constraints to the use of this substance in the medical field.

Industrially produced methylene blue also has the problem of some organic impurities resulting from the demethylation of dimethylamine groups present in position 3 and 5 in the phenothiazine structure. Such impurities are commonly referred to as “Azure” and are depicted below:

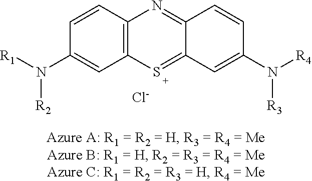

The use of methylene blue in the medical field requires a limitation of both metal impurities and of organic impurities, as defined by the current Pharmacopoeias (e.g. European Pharmacopoeia and United States Pharmacopeia).

The purification of methylene blue from metal impurities and from Azure A-C above has no simple resolution due to the sequestering effect of the diaminophenothiazinium structure and to the chemical analogy of organic contaminants. The same applies to the purification of Azure A-C above that are used as dyes in diagnostics.

The European Patent application n. 3458522 discloses a preparation of methylene blue and its purification to reduce those impurities.

However, said Patent application is silent about another impurity to be desirably removed from methylene blue, i.e. N-nitroso-Azure B having formula shortly referred to as “NnAB”:

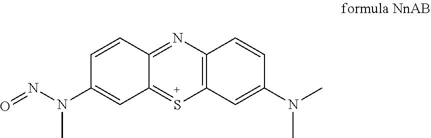

It is therefore an object of the present invention to provide a methylene blue which is essentially free of N-nitroso-Azure B, while being similarly free of metal impurities, Azure A and Azure C.

### SUMMARY OF THE INVENTION

Said object has been achieved by a methylene blue comprising less than 0.7 ppm of N-nitroso-Azure B.

The present invention also concerns a process for the preparation of said methylene blue.

Moreover, the present invention concerns a pharmaceutical composition comprising said methylene blue.

In another aspect, the present invention concerns said methylene blue for use in the treatment of methemoglobinemia.

In a further aspect, the present invention concerns a method of analysis to determine the level of N-nitroso-Azure B in the methylene blue, the method being performed via HPLC, column chromatography, paper chromatography, thin-layer chromatography, liquid chromatography, affinity chromatography, ion exchange chromatography, size-exclusion chromatography, reversed-phase chromatography, titration, NMR, LCMS, or LC-ESI-HRMS.

### BRIEF DESCRIPTION OF THE FIGURES

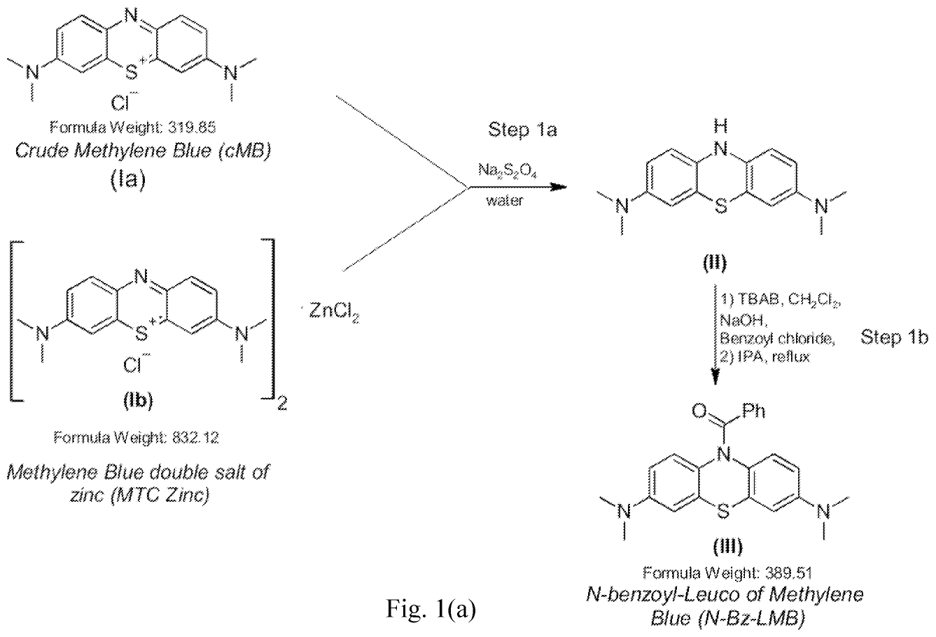

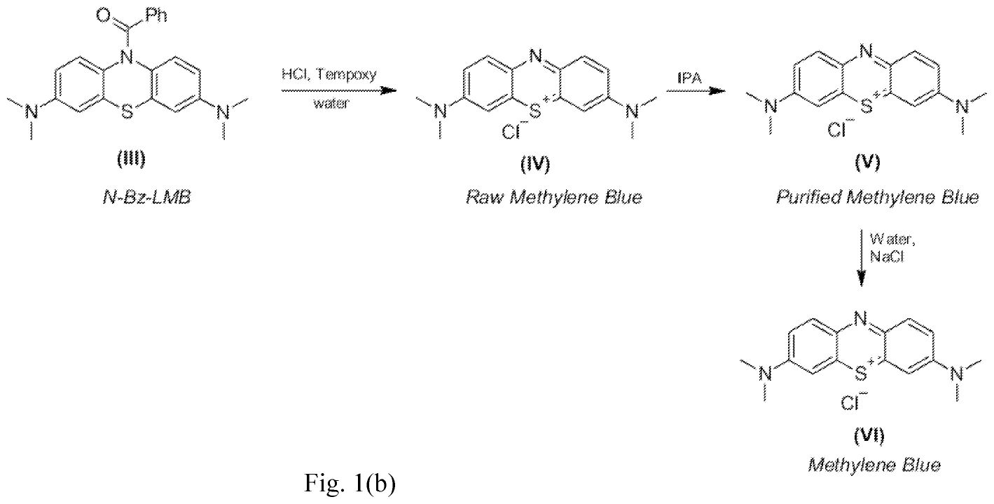

The characteristics and the advantages of the present invention will become apparent from the following detailed description, from the working examples provided for illustrative purposes, and from the annexed FIGS. 1(a) and 1(b) showing a preparation scheme of the invention, according to a preferred embodiment.

### DETAILED DESCRIPTION OF THE INVENTION

The invention therefore relates to methylene blue comprising ≤0.7 ppm (i.e. equal to or less than 0.7 ppm) of N-nitroso-Azure B having formula NnAB:

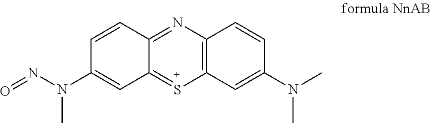

The content of impurity N-nitroso-Azure B (also known as ‘N-Nitroso-desmethyl-methylene blue impurity’—NDSRI) in the methylene blue can be determined by HPLC, column chromatography, paper chromatography, thin-layer chromatography, liquid chromatography, affinity chromatography, ion exchange chromatography, size-exclusion chromatography, reversed-phase chromatography, titration, NMR, LCMS/MS, or LC-ESI-HRMS.

Preferably, the determination of N-nitroso-Azure B content in the methylene blue is performed by LC-MS/MS.

In preferred embodiments, the determination of N-nitroso-Azure B content in the methylene blue is performed by LC-MS/MS, wherein the stationary phase in the column is silica gel, and the mobile phase is firstly a mixture of formic acid and water, and secondly a mixture of acetonitrile and methanol.

In view of the fact that the methylene blue comprises ≤0.7 ppm of NnAB, the methylene blue of the present invention is defined to be “essentially free of N-nitroso-Azure B”.

Preferably, methylene blue of the present invention comprises less than 0.1 ppm of N-nitroso-Azure B having formula NnAB.

More preferably, methylene blue of the present invention comprises less than 0.07 ppm of N-nitroso-Azure B having formula NnAB. The methylene blue of these embodiments is defined to be “free of N-nitroso-Azure B”.

Methylene blue of the present invention also features an overall purity of greater than 95%, preferably greater than 97%, more preferably greater than 99%, and most preferably greater than 99.9%, as measured by LC-MS/MS. For these reasons, the methylene blue of the present invention is also referred to as “highly pure”.

In preferred embodiments, methylene blue of the present invention features an overall purity of 99% to 99.95%, more preferably 99.5% to 99.99%.

Methylene blue of the present invention also features an overall heavy metal content within the limits set by the European Pharmacopoeia 8.0, and anyway not higher than 1,000 ppm, as measured by HPLC, where heavy metals are cadmium, mercury, arsenic, tin, manganese, molybdenum, nickel, lead, chromium, aluminium, zinc, iron, copper, and sodium.

In preferred embodiments, methylene blue of the present invention is free of N-nitrosamines (N-NAs) selected from N-methyl-N-phenylnitrous amide (NMPA), N-methyl-N-(4-nitrosophenyl)nitrous amide (NMPNA), N-(4-aminophenyl)-N-methyl nitrous amide (NMPMNA), N-methyl-N-(p-tolyl)nitrous amide (NMNMA), and mixtures thereof. N-NAs is a class of nitrogen-containing impurities, that share a common nitrosamino functional group, to which N-nitroso-Azure B also belongs. With the term “free of”, it is meant to define that those impurities are non-detected via LC-MS, each being below 0.03 ppm, as the limit of detection.

In preferred embodiments, methylene blue of the present invention comprises less than 100 ppm of potentially genotoxic impurities (PGIs) selected from N,N-dimethyl aniline (NNDMA), N,N-dimethyl-4-nitroso aniline (NNDM-p-NO), N-methylaniline (NMA), p-amino-dimethyl aniline thiosulfonic acid (ADMA-TSA), bis-(4-dimethylaminophenyl)amine (B-DMAPA) [also known as Bindschedler's green leuco base or BGL], p-amino-dimethyl aniline (ADMA), and mixtures thereof, as measured via HPLC. More preferably, methylene blue of the present invention comprises less than 50 ppm of PGIs.

In preferred embodiments, methylene blue of the present invention is free of PGIs, as each PGI is non-detected via HPLC, being below the respective limit of detection (LOD), i.e. 5 ppm LOD for NNDMA, 2 ppm LOD for NNDM-p-NO, 5 ppm LOD for ADMA-TSA, and 2.5 ppm LOD for B-DMAPA, or being below the respective limit of quantification (LOQ), i.e. below 25 ppm LOQ for NMA and below 25 ppm LOQ for ADMA.

In the most preferred embodiments, methylene blue of the present invention is free of N-NAs and comprises less than 100 ppm of PGIs. It should be understood that any combination of preferred sub-ranges above reported is hereby disclosed and even more preferred accordingly.

The methylene blue of the invention can be prepared by the following process comprising the steps of:
1) providing a starting methylene blue selected from crude methylene blue (Ia) (crude MB), methylene blue double salt of zinc (Ib) (MB zds), and mixtures thereof,
2) adding a reducing agent, and subsequently a protective agent comprising a functional group -Pr to obtain a compound of formula III:

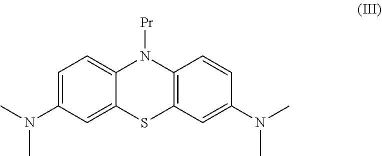

wherein the protective agent is selected from benzoyl chloride, benzoic anhydride, methylbenzoyl chloride, benzoyl bromide, methylbenzoyl bromide, dimethylbenzoyl chloride, chlorobenzoyl chloride, dichlorobenzoyl chloride, methoxybenzoyl chloride, nitrobenzoyl chloride, acetyl chloride, acetic anhydride, propionyl chloride, propionic anhydride, di-tert-butyl dicarbonate, benzylchloroformiate, ethylchloroformiate and mixtures thereof,
3) isolating the resulting compound of formula III, adding isopropyl alcohol and heating at reflux, then cooling and filtering at 0-5° C., while washing with isopropyl alcohol,
4) oxidating the compound of formula (III) resulted from step 3), by adding a 15-25% solution of a stable free radical agent over a period of time of 2-2.5 h at 30-35° C., then stirring for 40-50 minutes, to obtain a raw methylene blue, said stable free radical agent being NHAc-TEMPO, 4-C1-6-alkyloxy-TEMPO, 4-benzoxyloxy-TEMPO, 4-methoxy-TEMPO, 4-carboxylic-4-amino-TEMPO, 4-chloro-TEMPO, 4 hydroxylimine-TEMPO, 4-hydroxy-TEMPO, 4-oxo-TEMPO, 4-oxo-TEMPO-ethylene ketal, 4-amino-TEMPO, or a mixture thereof,
5) adding a water solution of NaCl, stirring for 2-4 h at room temperature, filtering off the raw methylene blue so obtained, then washing with diluted HCl, and
6) triturating with isopropyl alcohol the raw methylene blue, filtering off, washing with isopropyl alcohol, and then crystallizing from a water solution of NaCl, wherein no sequestering agent is used in the process steps.

In step 1), a starting methylene blue is provided, being selected from crude methylene blue (Ia), methylene blue double salt of zinc (Ib) (shortly ‘MB zds’), and mixtures thereof. “Crude” methylene blue denotes a methylene blue isolated and washed, but not yet subjected to a purification.

In step 2), a reducing agent is added, and subsequently a protective agent comprising a functional group -Pr to obtain a compound of formula III.

Preferably, said reducing agent is selected from sodium dithionite, sodium borohydride, methylhydrazine, hydrazine, hydrazine hydrate, ascorbic acid, formic acid, hydrogen, oxalic acid, dithiothreitol, phosphites, hypophosphites and mixtures thereof.

In preferred embodiments, said reducing agent is sodium dithionite.

In preferred embodiments, said protective agent is benzoyl chloride.

Preferably, step 2) is performed at temperatures not higher than 35° C., for 35-40 minutes.

Preferably, said starting methylene blue and said reducing agent are in an equivalent (eq.) ratio of 1:1.1 to 1:1.5.

Preferably, said starting methylene blue and said protective agent are in an equivalent (eq.) ratio of 1:2 to 1:3.

Preferably, step 2) is a one-pot synthesis step. This means that the reaction of the starting methylene blue with a reducing agent and a protective agent to obtain a compound of formula III takes place in a single reaction environment, or in a single container or reactor.

In this way, it is possible to avoid using long processes of separation and purification of the intermediates products obtained, thereby saving not only time but also resources (equipment, chemicals, solvents, etc.). Moreover, as a result, the chemical yield can be increased, having reduced intermediate substance losses.

In step 3), the compound of formula III is isolated, isopropyl alcohol is added and the resulting mixture heated at reflux, then cooled and filtered at 0-5° C., while washing with isopropyl alcohol.

Preferably, the compound of formula III is isolated by phase separation, washing with water and distillation.

In step 4), the compound of formula (III) resulted from step 3) is oxidated, by adding a 15-25% solution of a stable free radical agent over a period of time of 2-2.5 h at 30-35° C., then stirred for 40-50 minutes, to obtain a raw methylene blue.

Preferably, said stable free radical agent is TEMPO, 4-hydroxy-TEMPO, 4-oxo-TEMPO, or 4-amino-TEMPO.

More preferably, said stable free radical agent is 4-hydroxy-TEMPO (also called Tempoxy).

More preferably, the compound of formula (III) and said stable free radical agent are in an equivalent (eq.) ratio of about 1:2.

Preferably, said stable free radical agent is the only oxidizing agent used in step 4) of the process of the invention.

In step 5), a water solution of NaCl is added, stirred for 2-4 h at room temperature, to remove the protective group -Pr; the raw methylene blue so obtained is filtered off, then washed with diluted HCl.

For the purposes of the present invention, “room temperature” means a temperature of 20-25° C.

In step 6), the raw methylene blue is triturated with isopropyl alcohol, filtered off, washed with isopropyl alcohol, and then crystallized from a water solution of NaCl.

Preferably, the raw methylene blue is triturated with isopropyl alcohol, stirred for 2-4 h at room temperature, then filtered off and washed with isopropyl alcohol.

Preferably, the methylene blue so washed is dissolved in a water solution of NaCl at 70-85° C., filtered through a lenticular filter, and washed with water, then cooled down to 25-30° C., stirred for 8-16 h, centrifugated and washed with diluted NaCl.

Preferably, steps 3) to 6) are performed in a one-pot. This means that all these steps take place in a single reaction environment, or in a single container or reactor.

Remarkably, the process of the invention requires no sequestering agent, such as EDTA and derivatives thereof to remove heavy metals from methylene blue.

It was surprisingly found that the process of the invention allows to obtain methylene blue essentially free of N-Nitroso-Azure B, at the same time featuring an overall impurity content equal or less than 3%, as well as an overall heavy metal content of 25-1,000 ppm.

This means that the process achieves an unexpected improvement in reducing a specific impurity, such as N-Nitroso-Azure B, while maintaining high performances in reducing other impurities, such as Azure A-C and heavy metals. Moreover, the steps can be performed in one-pot, i.e. mostly without separating the reaction intermediates, with evident advantages in terms of overall speed and environmental and human safety.

The starting methylene blue of step 1) can be a commercially available product.

Otherwise, the latter may be prepared using the following synthetic route.

The synthesis of MB zds and crude MB provides for the construction of the MB tricyclics structure using N,N-dimethylaniline as a starting material, which is initially converted to N,N-dimethyl-4-nitrosoaniline by treatment with NaNO₂ in aqueous HCl at low temperatures.

The nitrous group of N,N-dimethyl-4-nitrosoaniline is reduced to amino group by treatment with iron powder and HCl at low temperatures. Excess iron is removed by filtration and the mother liquors containing N,N-dimethyl-p-phenylenediamine hydrochloride are directly used in the next step.

Four solutions containing aluminium sulphate octadecahydrate, sodium thiosulfate, sodium dichromate, and HCl, are sequentially added to the waters of the previous step.

The reaction mixture results in Intermediate 1:

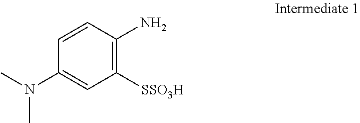

which is used directly in the next step without further processing.

Two solutions containing N,N-dimethylaniline, sodium dichromate, and H₂SO₄ are sequentially added to the mixture containing Intermediate 1, thus resulting in a reaction mixture containing Intermediate 2:

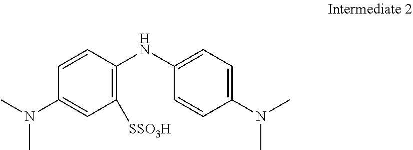

which is used directly in the next step without further processing.

Copper sulphate is added to the mixture containing Intermediate 2 and it is heated (85-95° C.) to allow the final cyclization, then let to cool down to 50-55° C. H₂SO₄ (50%) is then poured onto the resulting product, while keeping the same temperature, with the aim to remove chromium salts. At the end of this pouring step, the temperature is left to reach 20-25° C., while the product is maintained under stirring.

The precipitate is recovered by filtration and dissolved in hot water.

Crude methylene blue is precipitated from the solution by adding sodium chloride, which is then crystallized by water.

When the starting methylene blue is prepared according to the synthesis above described, the methylene blue obtained by the process of the present invention is advantageously free of N-nitrosamines (N-NAs) and comprises less than 100 ppm of potentially genotoxic impurities (PGIs).

The present invention also concerns a pharmaceutical composition comprising the methylene blue above described, i.e. methylene blue comprising ≤0.7 ppm of N-nitroso-Azure B having formula NnAB, and pharmaceutically acceptable excipients.

Preferably, the whole pharmaceutical composition of the present invention comprises ≤0.7 ppm of N-nitroso-Azure B having formula NnAB.

In another aspect, the present invention concerns the methylene blue or the pharmaceutical composition as above described, for use in the treatment of methemoglobinemia.

In other words, there is provided a method of treating methemoglobinemia, the method comprising the step of administering to the patient in need thereof of a therapeutically effective amount of methylene blue comprising ≤0.7 ppm of N-nitroso-Azure B having formula NnAB.

Alternatively, there is provided a use of methylene blue for the manufacture of medicament for use in the treatment of methemoglobinemia, wherein the methylene blue comprises ≤0.7 ppm of N-nitroso-Azure B having formula NnAB.

In a further aspect, the present invention concerns a method of analysis to determine the level of N-nitroso-Azure B in the methylene blue or in the pharmaceutical composition containing the same, the method being performed via HPLC, column chromatography, paper chromatography, thin-layer chromatography, liquid chromatography, affinity chromatography, ion exchange chromatography, size-exclusion chromatography, reversed-phase chromatography, titration, NMR, LCMS, or LC-ESI-HRMS.

In preferred embodiments, said method of analysis to determine the level of N-nitroso-Azure B comprises the steps of:
(i) subjecting a methylene blue sample, or pharmaceutical composition sample, to LC-MS/MS technique, wherein the stationary phase in the column is silica gel, and the mobile phase is firstly a mixture of formic acid and water, and secondly a mixture of acetonitrile and methanol;
(ii) determining the presence of N-nitroso-Azure B in the sample; and
(iii) quantifying the amount of N-nitroso-Azure B.

In more preferred embodiments, in step (i), the column is Intersil ODS-3 (100 mm×4.6 mm; 3 μm).

It should be also understood that all the combinations of preferred aspects of the methylene blue of the invention, as well as of the preparation processes, and pharmaceutical compositions of the same, as above reported, are to be deemed as hereby disclosed.

All combinations of the preferred aspects of the methylene blue of the invention, preparation processes, and pharmaceutical compositions disclosed above are to be understood as herein described.

Below are working examples of the present invention provided for illustrative purposes.

### EXAMPLES

### LC-MS/MS Methods Used for Detecting and Quantifying N-Nitroso-Azure B in Methylene Blue

### Instruments and Materials

LC-MS: Sciex API 4000
Column: Inertsil ODS-3 (100 mm×4.6 mm), 3 μm

### Test Methodology

Mobile phase A: 1.0 mL formic acid in 1000 mL of purified water
Mobile Phase B: acetonitrile and methanol in the ratio of (50:50) % v/v
Diluent: 1.0 mL of formic acid in mixer of methanol and purified water in the ratio of (50:50) % v/v.
Standard preparation: Transferred an accurately weighed quantity of about 7.1 mg of N-nitroso-Azure B standard into a 10 mL volumetric flask. Made volume up to the mark with diluent and mix.
Diluted 1.0 mL of this solution to 250.0 mL with diluent and mixed.
Diluted 1.0 mL of this solution to 200.0 mL with diluent and mixed.
Sensitivity solution: Diluted 2.5 mL of N-nitroso-Azure B standard preparation to 10 mL volumetric flask with diluent and mixed well.

### Chromatographic System

Column: Inertsil ODS-3 (100 mm×4.6 mm) 3 μm
Flow rate: 0.65 mL/minute
Injection volume: 50 μL
Column temperature: 55° C.
Auto sampler temperature: 5° C.
Run Time: 15 minutes

Gradient program

| Time (in minutes) | % Mobile phase A | % Mobile phase B |
|---|---|---|
| 0 | 70 | 30 |
| 5 | 50 | 50 |
| 10 | 10 | 90 |
| 10.5 | 70 | 30 |
| 15 | 70 | 30 |

### Auto Sampler Parameters for Shimadzu HPLC (Model: Prominence SIL-HTC)

Rinsing volume: 200 μL
Needle stroke: 52 mm
Rinsing speed: 35 μL/sec
Sampling speed: 5 μL/sec
Rinse dip time: 15 sec
Rinse Mode: Before and after aspiration

### Mass Parameters (Sciex API 4000 or Equivalent Instrument)

Scan type: MRM Mode
Ionization mode: ESI
Scheduled MRM: No
Polarity: Positive
Resolution Q1: Unit
Resolution Q3: Unit
Dwell (msec): 200

### Limit of Detection

LOD: 0.058 ppm

### Hplc Methods Used for Detecting and Quantifying NNDMA and NNDM-p-NO in Methylene Blue

Column: CPS Hypersil-2 5μ 250×4.6 mm
Flow: 1.3 ml/min
Mobile Phase A: Buffer NH₄OOCCH₃ 10 mM, pH=6.8±0.05, eventually adjusted with diluted NH₃ or CH₃COOH
Mobile Phase B: CH3OH/CH3CN 75:25

Gradient program

| time (min) | % Mobile phase A | % Mobile Phase B |
|---|---|---|
| 0 | 65 | 35 |
| 6.5 | 65 | 35 |
| 15 | 5 | 95 |
| 35 | 5 | 95 |
| 36 | 65 | 35 |
| 45 | 65 | 35 |

Run time: 40 min
Injection Volume: 10 μl
Column Temperature: 35° C.
UV-VIS Detector: 242 nm and 400 nm
Diluent: Methanol
Instrument used for this validation: Agilent 1290 infinity
Column Wash: At the end of the analysis wash the column at 1.0 ml/min with a 60:40 H₂O/CH₃OH for about 40 minutes and further 40 min with H₂O/CH₃CN 20:80.
Retention times registered on HPLC instrument Agilent 1290 model and working detector wavelengths of impurities:

| Impurity | RT (min) | Detection Wavelength (nm) |
|---|---|---|
| NNDM-p-NO | 3.39 | 400 |
| NNDMA | 4.17 | 242 |

Retention time Methylene Blue: 35-40 min
Sample Concentration: 20 mg/ml.
N,N-Dimethylaniline (NNDMA), LOD: 5 ppm
N,N-Dimethyl-p-nitroso aniline (NNDM-p-NO), LOD: 2 ppm
N-Methylaniline (NMA) was validated only as limit test using the same chromatographic conditions (242 nm). LOD: 50 ppm
   Hplc Methods Used for Detecting and Quantifying p-Amino-Dimethylaniline Thiosulfonic Acid and Bindschedler's Green Leuco Base in Methylene Blue
Instrument: HPLC Agilent 1260 series with UV detector or equivalent
Detector wavelength: 210 and 290 nm
Column: Zorbax SB-Phenyl 5μ 250×4.6 mm
Column temperature: 35° C.
Mobile phase A: 0.1% v/v HClO₄ (70% Perchloric acid)
Mobile phase B: Acetonitrile
Diluent: Mobile phase A/Methanol 1:1
Flow rate: 1.3 mL/min
Injection volume: 20 μL
Run time: 40 minutes

Gradient program:

| Time (minutes) | Mobile Phase-A | Mobile Phase-B |
|---|---|---|
| 0 | 90 | 10 |
| 30 | 30 | 70 |
| 32 | 30 | 70 |
| 33 | 90 | 10 |
| 40 | 90 | 10 |

Retention times:

| Impurity | RT (min) | Channel | |
|---|---|---|---|
| ADMA-TSA | 3.4 | 210 | nm |
| BGL | 6.6 | 290 | nm |
| Methylene Blue | 16-21 | 210/290 | nm |

### Procedure

Diluent: Mobile phase A/Methanol 1:1
Stock Standard solution: Accurately weigh and transfer about 50 mg of ADMA-TSA and BGL standards into a 200 mL volumetric flask. Dissolve and dilute to volume with diluent.
Nominal concentration 0.25 mg/mL.

### Intermediate Solution:

Dilute 1.0 mL of Stock Standard solution to 25 mL with diluent.
Nominal concentration 0.01 mg/mL.

### Standard Solution:

Into 20 mL volumetric flask, weight accurately about 200 mg of Methylene Blue and dissolve with about 10 mL of diluent, then add 1.0 mL of Intermediate solution and bring to volume with diluent.
Nominal concentration of sample 10 mg/mL. Nominal concentration of impurities 0.0005 mg/mL (50 ppm).

### Sample Solution:

Accurately weigh and transfer about 200 mg of sample into a 20 mL volumetric flask.

Dissolve and dilute to volume with diluent.

| Solution | N° inj |
|---|---|
| Blank | ≥1 |
| STD solution | 6 |
| Sample solution | 1 |
| STD solution | 1 |

p-Amino-dimethylaniline thiosulfonic acid (ADMA-TSA)-LOD: 5 ppm
Bindschedler's green leuco base (C.A.S.: 637-31-0)-LOD: 2.5 ppm
   Hplc Methods Used for Detecting and Quantifying p-Amino-Dimethylaniline in Methylene Blue
Chromatographic conditions
Instrument: HPLC Agilent 1260 series with UV detector or equivalent
Detector wavelength: 246 nm
Column: Zorbax SB-CN 5μ 250×4.6 mm
Column temperature: 50° C.
Buffer: 1.8 g Disodium phosphate dodecahydrate, 2.8 g of Potassium dihydrogen phosphate and 1 g of Heptane 1-sulfonic acid sodium salt in 1 L of purified water.
Mobile phase A: Buffer:Acetonitrile (90:10)
Mobile phase B: Acetonitrile:Water (90:10)
Diluent: Acetonitrile/Methanol 1:1
Flow rate: 1.0 mL/min
Injection volume: 5 μL
Run time: 90 minutes

Gradient program:

| Time (minutes) | Mobile Phase-A | Mobile Phase-B |
|---|---|---|
| 0 | 100 | 0 |
| 17 | 100 | 0 |
| 18 | 5 | 95 |
| 55 | 5 | 95 |
| 56 | 100 | 0 |
| 90 | 100 | 0 |

Retention times:

| Impurity | RT (min) |
|---|---|
| p-amino-dimethylaniline | 8.8 |
| Methylene Blue | 33-44 |

### Procedure

Diluent: Methanol/Acetonitrile: 20/80
Stock Standard solution: Accurately weigh and transfer about 50 mg of p-amino-dimethylaniline standard into a 200 mL volumetric flask.
Dissolve and dilute to volume with diluent.
Nominal concentration: 0.25 mg/mL.
Standard solution: Dilute 1.0 mL of Stock Standard solution to 50 mL with diluent.
Nominal concentration: 0.005 mg/mL (250 ppm).
Sample solution: Accurately weigh and transfer about 200 mg of sample into a 10 mL volumetric flask. Dissolve and dilute to volume with diluent. Nominal sample concentration 20 mg/mL

| Solution | N° inj |
|---|---|
| Blank (FM) | 1 |
| Blank (Diluent) | 1 |
| STD solution | 6 |
| Sample solution | 1 |
| STD solution | 1 |

LOQ of the p-amino-dimethylaniline is 25 ppm.

### Example 1. Preparation of Crude Methylene Blue

In a 2 L 4-neck flask, kept under nitrogen, 300 ml of water was charged. The mixture was cooled to 0-5° C. with an ice bath and N,N-Dimethylaniline (40 g) was added. The loading funnel was washed with 80 ml of water. Maintaining the temperature at 0-10° C., 37% HCl (90 ml, 1.08 moles, 3.3 eq) was dropped down over 15-20 minutes, then the loading funnel was washed with 20 ml of water the mixture was stirred at 0-5° C. for approximately 1 h.

In a 500 mL flask, sodium nitrite (23.5 g) and 200 ml of purified water were loaded. The obtained solution was transferred to a loading funnel. This sodium nitrite solution was added into the flask containing the N,N-dimethylaniline, maintaining the temperature at 0-10° C. The mixture was stirred at 0-5° C. for 1-1.5 hours.

37% HCl was added into the reaction (90 ml, 1.08 moles, 3.3 eq.), maintaining the temperature at 0-5° C. Keeping the flask under a nitrogen flow and starting from 0-5° C., remaining in the range of 0-15° C., powdered iron was added in portions (total: 48 g; 2.6 eq.). Once the iron was added, the temperature was increased to 20-25° C. and the resulting mixture was stirred under a light flow of nitrogen for about 16 hours. The reaction was filtered on cardboard, washed with 80 ml of water, and the solid was recovered (“waste solid 1”). The filtered solution was used directly for the subsequent step.

The following solutions were prepared:
Water (90 ml) and sodium thiosulphate (90 g) were poured into a 500 ml flask and the stirring was maintained until complete dissolution was observed (NB: technical sodium thiosulphate is pentahydrate)
Water (142 ml) and a 61% aqueous solution of sodium dichromate at 61% (58 g, 0.13 moles, 0.4 eq; 17% solution) were poured into a 500 ml flask and stirred to obtain a homogeneous solution (24% solution).
N,N-dimethylaniline (40 g) was poured into a 500 ml flask; maintaining the temperature below 20° C., with an ice bath, 50% sulfuric acid (64 g) was poured into the same flask. The loading funnel used was washed with 8 ml of water.
Water (232 ml) and a 61% aqueous solution of sodium dichromate (156 g, 0.36 moles, 1.1 eq) were poured into a 500 ml flask (24% solution).
In a 100 ml flask, copper sulphate pentahydrate (11 g) and water (36 ml) were added and the mixture was stirred until complete dissolution was observed.

In a 3 14-neck flask, the solution obtained in the previous step containing the intermediate p-amino-dimethyl aniline was added.

Maintaining 20-25° C. and starting from the initially recorded pH (pH: 5.5), the pH was adjusted to the range 2.4-2.8 by slowly addition of 37% HCl (15 ml used; obtained: 2.73). Then Al2(SO4)3*18H2O (64 g) was added and the mass was cooled to 0-5° C.

Maintaining the temperature at 0-5° C., the solution of sodium thiosulphate pentahydrate (90 g in 90 ml of water) prepared previously was added, then the solution of 17% sodium dichromate (58 g of an aqueous solution of 61% sodium dichromate in 142 ml of water) was added in 20-30 minutes, always maintaining the temperature 0-5° C.

The mixture was stirred at 0-10° C. for 1 hour.

Maintaining 0-10° C., the solution of N,N-dimethylaniline in sulfuric acid was added, washing the dripping funnel used with water (10 ml).

Maintaining 0-10° C., the 24% sodium dichromate solution (156 g of 61% aqueous sodium dichromate solution in 230 ml of water) was added at approximately 20-30° C.

Once the addition was finished, the temperature was allowed to rise to 20-25° C. and stirring was continued overnight (16 hours).

The intermediate present into the mixture was Bindschedler green thiosulfonic acid, which was not isolated. The subsequent cyclization reaction was performed directly in the same reactor.

To the reaction mixture obtained previously, the copper sulphate solution (11 g in 36 ml of water) was added at 20-25° C., then the mixture was heat to 85-95° C. and stirred at this temperature for 1 hour, then the reaction mixture was cooled from the cycling temperature (85-95° C.) to the temperature of 50-55° C.

Maintaining 50-60° C. 50% sulfuric acid (220 g) was added and the temperature was allowed to drop slowly to 20-25° C.

The mass was stirred at this temperature for 3 hours, then it was filtered and the filter was washed with 100 ml of water (2 washes of 50 ml).

Wet Crude Raw Methylene Blue was recovered.

About 198 g of wet raw crude was isolated, equal to the theoretical calculated dry weight of 125 g. Weight yield compared to the starting 40 g of N,N-dimethylaniline: 312.5%

In a clean 3 L flask, all the Crude Raw Methylene Blue (approximately 125 g of dry equivalent) was charged.

Purified water (1500 ml) was added and the mass was heated to 88-95° C. and stirred for about 30 minutes at 88-95° C. This hot solution was filtered through a cloth filter while maintaining the temperature of the mass always at 88-95° C. directly inside another clean flask containing sodium chloride (400 g), retaining the “waste solid 2” on the cloth and keeping the hot filtered solution containing the product. The “waste solid 2” was washed with purified water pre-heated at 88-95° C., then it was recovered and discarded (approximately 81 g wet).

The filtered solution was slowly cooled (in about 1 hour) to 20-25° C. and stirred for 5 hours. The mass was filtered through a cloth filter and washed with a solution of NaCl 4 g/L (160 ml; 20-25° C.).

70.4 g of Wet Raw Methylene Blue were recovered, equal to the theoretical calculated dry weight of 45.6 g.

Weight yield compared to the starting 40 g of N,N-dimethylaniline: 114%.

### Example 2. Preparation of Methylene Blue

1) Synthesis of N-Benzoyl-Leuco-Methylene Blue (N-Bz-LMB) (See FIG. 1(a)):

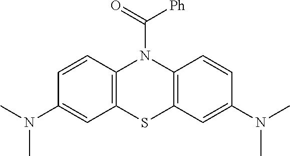

Water (2.5 L) and wet crude Methylene Blue produced as described in Example 1 (theoretical dry weight of 0.210 Kg; 0.6577 mol) were added to a proper flask. Under a nitrogen atmosphere, sodium dithionite (0.148 Kg; 1.3 eq) was added. The mixture was stirred for 45-60 minutes, then to the same flask was added TBAB (0.025 Kg; 0.12 eq) and methylene chloride (3.4 Kg). Keeping the temperature at 20-35° C., NaOH 30% (0.22 Kg, equal to 2.5 eq of NaOH) and benzoyl chloride (0.163 Kg; 1.7 eq) were added. The resulting mixture was stirred at 20-35° C. for about 12 h. The organic phase was separated and washed with a diluted aqueous solution of sodium dithionite. The organic phase was separated and the solvent was distilled off. Isopropyl alcohol (2.55 L) was added and heated up to reflux and maintained for about 1 h, then the mixture was cooled, filtered at about 3° C., washed with isopropyl alcohol and dried under vacuum, obtaining about 0.2125 Kg of N-Bz-LMB (82.95% molar yield). Average molar yield obtained: 73.8%

2) Oxidation of Methylene Blue (Briefly MB) (See FIG. 1(b)):

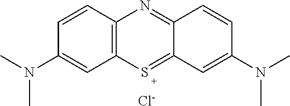

N-Bz-LMB (0.1642 Kg) and Water (1.478 L) were charged into a proper flask and the mixture was stirred for about 30 minutes. HCl 37% (1.008 Kg; 23.6 eq.) was added at 30-35° C. and then a 20 wt % aqueous solution of 4-hydroxy TEMPO (0.1452 Kg; 2 eq.) was added in about 2 h, keeping the temperature at 30-35° C. The mixture was stirred for about 40 minutes, then a solution of NaCl (0.156 Kg) in water (2.847 L) was added. The mixture was stirred at 20-25° C. for 2-4 h, then filtered and the solid was washed with 0.1N HCl.

The wet product and 1.314 L of isopropyl alcohol were added to a suitable flask, then stirred at room temperature for about 4 h, filtered off and finally washed with isopropyl alcohol (corresponding dried solid by LOD: 0.1237 Kg)

The resulting wet solid was dissolved in water (2.46 L) in the presence of NaCl (11.1 g), at 80° C., filtered through a lenticular filter, and washed with water. It was left to cool down to 25-30° C., stirred for 10 h, centrifugated and washed with aqueous solution of NaCl (2 g/L).

Average molar yield obtained from N-Bz-LMB: 90% MB

N-nitroso-Azure B (ppm): not-detected, according to the method above (LOD 0.058 ppm) Overall impurity content: 2.7-2.9% (via HPLC)

Azure B: 2.2% (via HPLC)

Overall heavy metal content: 25-1,000 ppm (via HPLC) [where heavy metals are cadmium, mercury, arsenic, tin, manganese, molybdenum, nickel, lead, chromium, aluminium, zinc, iron, copper, and sodium]

N-methyl-N-phenylnitrous amide (NMPA), N-methyl-N-(4-nitrosophenyl)nitrous amide (NMPNA), N-(4-aminophenyl)-N-methyl nitrous amide (NMPMNA), N-methyl-N-(p-tolyl)nitrous amide (NMNMA): not detected, via LC-MS (LOD 0.03 ppm)

Potentially genotoxic impurities (PGIs): not detected, via HPLC, being below the respective limit of detection (LOD), i.e. 5 ppm LOD for NNDMA, 2 ppm LOD for NNDM-p-NO, <25 ppm LOQ for NMA, 5 ppm LOD for ADMA-TSA, 2.5 ppm LOD for B-DMAPA, and <25 ppm LOQ for ADMA.

1. Methylene blue comprising ≤0.7 ppm of N-nitroso-Azure B, as measured by LC-MS/MS.

2. The methylene blue of claim 1 comprising less than 0.1 ppm of N-nitroso-Azure B.

3. The methylene blue of claim 1, being free of N-nitrosamines (N-NAs), as non-detected via LC-MS, said N-Nas being selected from N-methyl-N-phenylnitrous amide (NMPA), N-methyl-N-(4-nitrosophenyl)nitrous amide (NMPNA), N-(4-aminophenyl)-N-methyl nitrous amide (NMPMNA), N-methyl-N-(p-tolyl)nitrous amide (NMNMA), and mixtures thereof.

4. The methylene blue of claim 1, comprising less than 100 ppm of potentially genotoxic impurities (PGIs), as measured by HPLC, said PGIs being selected from N,N-dimethyl aniline (NNDMA), N,N-dimethyl-4-nitroso aniline (NNDM-p-NO), N-methylaniline (NMA), p-amino-dimethyl aniline thiosulfonic acid (ADMA-TSA), bis-(4-dimethylaminophenyl)amine (B-DMAPA), p-amino-dimethyl aniline (ADMA), and mixtures thereof; preferably less than 50 ppm of PGIs.

5. A pharmaceutical composition comprising the methylene blue of claim 1, and pharmaceutically acceptable excipients.

6. A process for preparing the methylene blue of claim 1, the process comprising the steps of:
1) providing a starting methylene blue selected from crude methylene blue (Ia) (crude MB), methylene blue double salt of zinc (Ib) (MB zds), and mixtures thereof,
2) adding a reducing agent, and subsequently a protective agent comprising a functional group -Pr to obtain a compound of formula III: 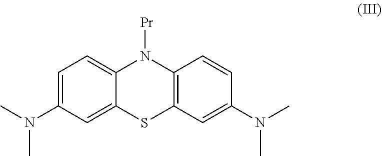
wherein the protective agent is selected from benzoyl chloride, benzoic anhydride, methylbenzoyl chloride, benzoyl bromide, methylbenzoyl bromide, dimethylbenzoyl chloride, chlorobenzoyl chloride, dichlorobenzoyl chloride, methoxybenzoyl chloride, nitrobenzoyl chloride, acetyl chloride, acetic anhydride, propionyl chloride, propionic anhydride, di-tert-butyl dicarbonate, benzylchloroformiate, ethylchloroformiate and mixtures thereof,
3) isolating the resulting compound of formula III, adding isopropyl alcohol and heating at reflux, then cooling and filtering at 0-5° C., while washing with isopropyl alcohol,
4) oxidating the compound of formula (III) resulted from step 3), by adding a 15-25% solution of a stable free radical agent over a period of time of 2-2.5 h at 30-35° C., then stirring for 40-50 minutes, to obtain a raw methylene blue, said stable free radical agent being NHAc-TEMPO, 4-C1-6-alkyloxy-TEMPO, 4-benzoxyloxy-TEMPO, 4-methoxy-TEMPO, 4-carboxylic-4-amino-TEMPO, 4-chloro-TEMPO, 4 hydroxylimine-TEMPO, 4-hydroxy-TEMPO, 4-oxo-TEMPO, 4-oxo-TEMPO-ethylene ketal, 4-amino-TEMPO, or a mixture thereof,
5) adding a water solution of NaCl, stirring for 2-4 h at room temperature, filtering off the raw methylene blue so obtained, then washing with diluted HCl, and
6) triturating with isopropyl alcohol the raw methylene blue, filtering off, washing with isopropyl alcohol, and then crystallizing from a water solution of NaCl,
wherein no sequestering agent is used in the process steps.

7. The process of claim 6, wherein step 2) is performed at temperatures not higher than 35° C., for 35-40 minutes, and the reducing agent is selected from sodium dithionite, sodium borohydride, methylhydrazine, hydrazine, hydrazine hydrate, ascorbic acid, formic acid, hydrogen, oxalic acid, dithiothreitol, phosphites, hypophosphites and mixtures thereof.

8. The process of claim 6, wherein the stable free radical agent is 4-hydroxy-TEMPO.

9. The process of claim 6, wherein in step 6), the raw methylene blue is triturated with isopropyl alcohol, stirred for 2-4 h at room temperature, then filtered off and washed with isopropyl alcohol, then the methylene blue so washed is dissolved in a water solution of NaCl at 70-85° C., filtered through a lenticular filter, and washed with water, then cooled down to 25-30° C., stirred for 8-16 h, centrifugated and washed with diluted NaCl.

10. A method of treating methemoglobinemia, the method comprising the step of administering to the patient in need thereof of a therapeutically effective amount of methylene blue comprising ≤0.7 ppm of N-nitroso-Azure B having formula NnAB.

11. A method of analysis to determine the level of N-nitroso-Azure B in methylene blue or in a pharmaceutical composition containing said methylene blue, the method comprising the steps of:
(i) subjecting a methylene blue sample, or a pharmaceutical composition sample, to LC-MS/MS technique, said technique comprising a column with a stationary phase of silica gel, and a mobile phase comprising first a mixture of formic acid and water, and second a mixture of acetonitrile and methanol;
(ii) determining the presence of N-nitroso-Azure B in the sample; and
(iii) quantifying the amount of N-nitroso-Azure B.

12. The method of analysis of claim 11, wherein, in step (i), the column is Intersil ODS-3 (100 mm×4.6 mm; 3 μm).
